(19) United States
(12) Patent Application Publication
(10) Pub. No.: US 2015/0320377 A1
(43) Pub. Date: Nov. 12, 2015

(54) MEDICAL IMAGE DISPLAY CONTROL APPARATUS, METHOD, AND PROGRAM

(71) Applicant: FUJIFILM Corporation, Tokyo (JP)

(72) Inventor: Jun MASUMOTO, Tokyo (JP)

(73) Assignee: FUJIFILM CORPORATION, Tokyo (JP)

(21) Appl. No.: 14/801,183

(22) Filed: Jul. 16, 2015

Related U.S. Application Data
(63) Continuation of application No. PCT/JP2014/000078, filed on Jan. 10, 2014.

(30) Foreign Application Priority Data
Jan. 30, 2013 (JP) 2013-014943

(51) Int. Cl. A61B 6/00; G06T 3/00; A61B 5/00; A61B 6/03; A61B 5/055

(52) U.S. Cl. CPC A61B 6/463; A61B 6/03; A61B 6/503; A61B 6/42; A61B 6/466; A61B 5/055; A61B 5/743; G06T 3/0068

(57) ABSTRACT

A medical image display control apparatus includes a medical image obtaining unit that obtains a set of time series medical images captured by successively imaging the same subject, an observation position obtaining unit that obtains anatomically common positions in the set of medical images as observation positions, and a display control unit that successively displays the set of medical images such that the observation positions in the set of medical images are displayed at the same position on a display screen.

### CROSS-REFERENCE TO RELATED APPLICATIONS

This application is a Continuation of PCT International Application No. PCT/JP2014/000078 filed on Jan. 10, 2014, which claims priority under 35 U.S.C. §119 (a) to Japanese Patent Application No. 2013-014943 filed on Jan. 30, 2013. Each of the above application(s) is hereby expressly incorporated by reference, in its entirety, into the present application.

### BACKGROUND OF THE INVENTION

1. Technical Field

The present invention relates to a medical image display control apparatus, method, and program for displaying a set of time series medical images obtained by successively imaging the same subject.

2. Background Art

The recent remarkable performance improvement in medical image capturing systems, including computed tomography (CT) and magnetic resonance (MR) has made possible to obtain not only a three-dimensional image captured at a particular time but also a four-dimensional image which also incorporates time axis information.

One of the most clinically significant aspects of the time axis information is motion analysis. In the field of heart, in particular, more than ten three-dimensional images can be obtained during a single beat, which may be displayed as volume-rendered images and further as an animation picture by successively displaying the images, whereby the beating of the heart can be observed in detail.

Generally, in a case where an entire heart is observed, the observation can be made efficiently by extracting the region of the heart in advance by image processing and displaying only the region of the heart. Further, a clipping function is often used for easy observation of a tissue within a heart. The clipping function virtually disposes a rectangular solid, a sphere, or a specific plane in a three-dimensional space, and visualizes only the inside or outside the shape, or only far side or front side of the shape, whereby a virtually cut observation target tissue can be displayed.

### SUMMARY OF INVENTION

Here, for the observation of a myocardium of a hear, for example, the observation using the clipping function described above is suitable but for the observation of a valve of a heart, for example, the motion of the valve cannot be observed in detail unless the valve is observed from the above.

Recently, aortic valve replacement surgery has been conducted frequently centering on the United States and the observation of heart valves has been getting important. The aortic valve replacement surgery replaces an aortic valve, which has highly calcified and no longer functions normally, with an artificial valve, and it is necessary to image the heart by a CT system or the like prior to the surgery to observe the calcification adjacent to the valve of the heart and the motion of the valve before the surgery.

Therefore, it is conceivable, for example, to animation-display a set of time series images obtained by imaging a beating heart for observing the motion of the valve. Then, it is conceivable to clip each image such that near the valve can be observed from the above when performing such animation-display. At this time, clipping the same range for each image, however, will result in that movement due to the beating of the heart is added to the motion of the valve itself, since the valve itself is moved by the beating of the heart, thereby causing a difficulty in observing only the motion of the valve.

U.S. Patent Application Publication No. 20120207365 extracts a path of an aorta in advance, sets a clipping area having a specific shape along the path, and displays only inside the clipping area, whereby the motion of only near the valve may be displayed.

But, the method described in U.S. Patent Application Publication No. 20120207365 cannot be said to be the optimal method to purely display only the motion of the valve, because the position of the clipping area is moved, also in that method, between the images of each phase.

Japanese Unexamined Patent Publication No. 2011-193997 proposes a display method which, when displaying a set of images captured by successively imaging a beating heart, facilitates observation of only the pure motion of the organ by eliminating an influence of a change in signal value between images of each phase, but proposes nothing about a display method that allows for the observation of only the motion of the valve described above.

In view of the circumstances described above, it is an object of the present invention to provide a medical image display control apparatus, method, and program which, when displaying a set of time series medical images obtained by successively imaging the same subject, allows for effective observation of local dynamics.

A medical image display control apparatus of the present invention includes a medical image obtaining unit that obtains a set of time series medical images captured by successively imaging the same subject, an observation position obtaining unit that obtains anatomically common positions in the set of medical images as observation positions, and a display control unit that successively displays the set of medical images such that the observation positions in the set of medical images are displayed at the same position on a display screen.

In the foregoing medical image display control apparatus, the observation position obtaining unit may be made to receive designation of the observation positions in the set of medical images.

Further, the observation position obtaining unit may be made to automatically detect the observation positions in the set of medical images.

Further, the apparatus may include a registration unit that performs a non-rigid registration on the set of medical images.

Still further, the observation position obtaining unit may be made to receive designation of an observation position in one of the set of medical images and, based on the received observation position in the one medical image and a result of the non-rigid registration, to obtain an observation position in a medical image other than the one medical image.

Further, the display control unit may be made to successively display the set of medical images such that the observation positions in the set of medical images are displayed in the center position of the display screen.

Still further, an image obtained by imaging a heart, including a valve may be used as the medical image, the observation position obtaining unit may be made to obtain an observation position in the valve, and the display control unit may be made to display the medical image of the valve viewed from the above.

Further, the apparatus may include a clipping unit that clips the set of medical images.

Still further, the clipping unit may be made to receive a clipping shape and to perform the clipping in the received shape.

Further, the clipping unit may be made to dispose a specific plane in an image space and to clip only the far side of the plane.

Still further, the display control unit may be made to display a volume rendered or a surface rendered image as the medical image.

Further, the display control unit may be made to display a two-dimensional tomographic image as the medical image.

Still further, the two-dimensional tomographic image may have a thickness.

Further, the two-dimensional image may be a MIP image, a MinIP image, or a Raysum image.

Still further, an image captured by a CT system or a MR system may be used as the medical image.

Further, the display control unit may be made to successively display the set of medical images such that a relative position between a range clipped by the clipping unit and the observation position is maintained.

Still further, the display control unit may be made to successively display the set of medical images such that the observation positions do not move in a depth direction of the display screen.

A medical image display control method of the present invention includes the steps of obtaining a set of time series medical images captured by successively imaging the same subject, obtaining anatomically common positions in the set of medical images as observation positions, and successively displaying the set of medical images such that the observation positions in the set of medical images are displayed at the same position on a display screen.

A medical image display control program of the present invention causes a computer to function as a medical image obtaining unit that obtains a set of time series medical images captured by successively imaging the same subject, an observation position obtaining unit that obtains anatomically common positions in the set of medical images as observation positions, and a display control unit that successively displays the set of medical images such that the observation positions in the set of medical images are displayed at the same position on a display screen.

According to the medical image display control apparatus, method, and program, a set of time series medical images captured by successively imaging the same subject is obtained, then anatomically common positions in the set of medical images are obtained as observation positions, and the set of medical images are successively displayed such that the observation positions in the set of medical images are displayed at the same position on a display screen. This allows local dynamics adjacent to the observation position to be observed efficiently.

### BRIEF DESCRIPTION OF THE DRAWINGS

FIG. 1 is a block diagram of a medical image diagnosis support system that uses a first embodiment of the medical image display control apparatus, method, and program of the present invention, illustrating a configuration thereof.

FIG. 2 is a drawing for explaining a method for obtaining, in a medical image of a valve viewed from the above, an observation position of the valve.

FIG. 3 is a drawing for explaining a method for obtaining, in a medical image of a valve viewed from the side, an observation position of the valve.

FIG. 4 is a flowchart for explaining an operation of the medical image diagnosis support system that uses the first embodiment of the medical image display control apparatus, method, and program of the present invention.

A to K of FIG. 5 show each clipped image extracted from a set of medical images of an aortic valve viewed from the above.

FIG. 6 is a block diagram of a medical image diagnosis support system that uses a second embodiment of the medical image display control apparatus, method, and program of the present invention, illustrating a configuration thereof.

FIG. 7 is a flowchart for explaining an operation of the medical image diagnosis support system that uses the second embodiment of the medical image display control apparatus, method, and program of the present invention.

FIG. 8 shows a display example of observation position specifying images.

### DETAILED DESCRIPTION OF THE PREFERRED EMBODIMENTS

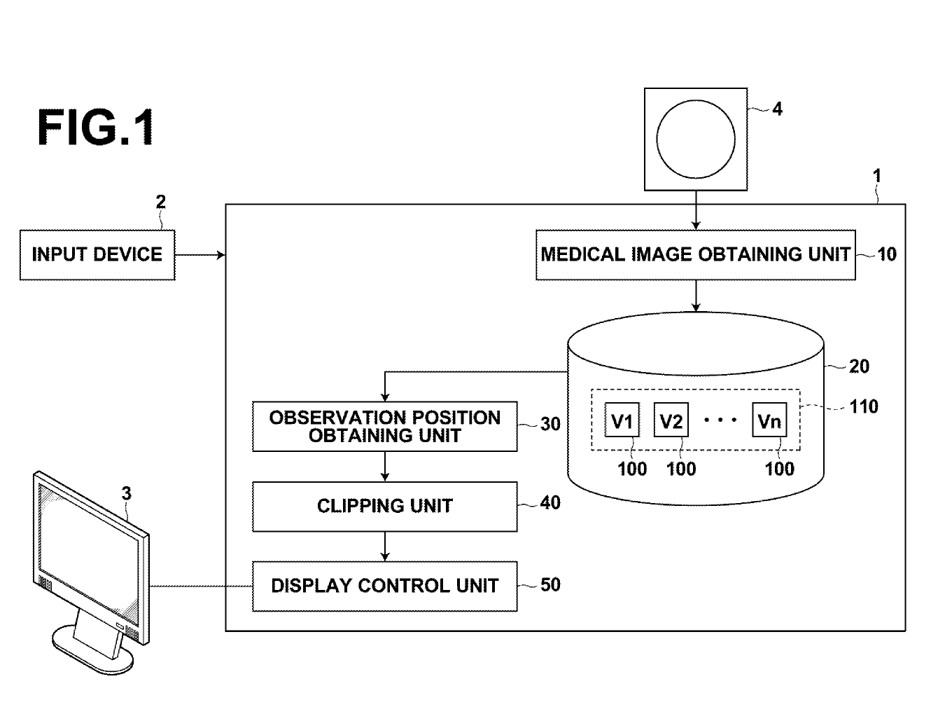

Hereinafter, a medical image diagnosis support system that uses a first embodiment of the medical image display control apparatus, method, and program of the present invention will be described with reference to the accompanying drawings. FIG. 1 is a block diagram of the medical image diagnosis support system that uses the present embodiment, illustrating a schematic configuration thereof.

As illustrated in FIG. 1, the medical image diagnosis support system of the present embodiment includes a medical image display control apparatus 1, an input device 2, and a display 3.

The medical image display control apparatus 1 is configured by installing a medical image display control program of the present embodiment on a computer. The medical image display control apparatus 1 includes a central processing unit (CPU) and storage devices, such as for example a semiconductor memory, a hard disk on which the medical image display control program of the present embodiment is installed, and a solid state drive (SSD). These pieces of hardware constitute a medical image obtaining unit 10, a medical image storage unit 20, an observation position obtaining unit 30, a clipping unit 40, and display control unit 50. Then, each unit operates when the medical image display control program installed on the hard disk is executed by the central processing unit.

The medical image obtaining unit 10 obtains a set of time series medical images 100 (V1 to Vn) captured by successively imaging the same subject. In the present embodiment, it is assumed that a set of a plurality of medical images 100 is obtained by imaging a beating heart in different phases. But, the present invention is not limited to the heart and other medical images of a specific organ, such as lung, may also be used.

The medical images 100 are those captured at a predetermined imaging interval At in the modality 4, such as a CT system, a MRI system, a Multi slice (MS) CT system, a cone beam CT system, an ultrasound imaging system, and a two-dimensional radiation image capturing system, and the medical images 100 may be tomographic images, volume data reconstructed from the tomographic images, two-dimensional fluoroscopic images, and the like.

Identification information that includes patient information and the type of modality are attached to a medical image group 110 constituted by a set of time series medical images 100, and the medical image group 110 obtained by the medical image obtaining unit 10 is stored in the medical image storage unit 20 with the identification information.

The medical image storage unit 20 is composed of a large capacity storage unit, such as a hard disk, and stores various medical image groups 110.

The observation position obtaining unit 30 obtains an anatomically common position in each medical image 100 constituting the medical image group 110 as an observation position. The observation position in each medical image 100 may be designated by the user using the input device 2, or a characteristic point in each medical image 100 may be detected automatically and the detected characteristic point may be obtained as the observation position.

The observation position may include, for example, the position of a point where each apex of the three valves constituting a tricuspid valve or an aortic valve of a heart intersects, and the like.

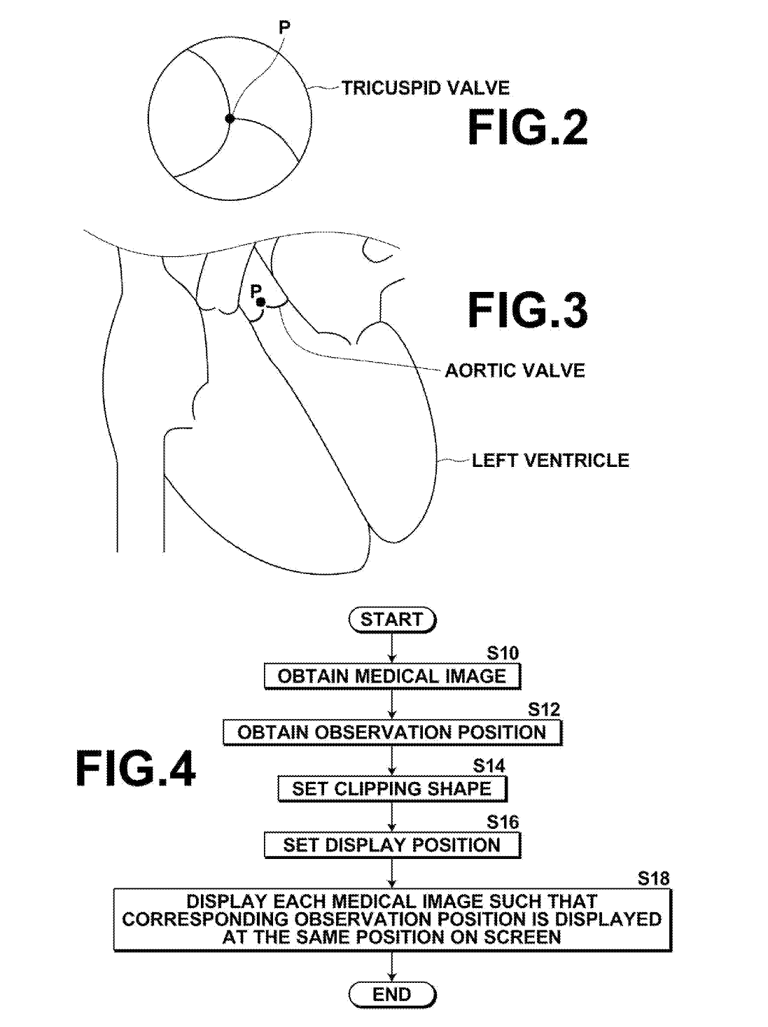

More specifically, a position P where the apex of each valve constituting a tricuspid valve or an aortic valve intersects substantially corresponds to the center of the tricuspid valve or the aortic valve, as illustrated in FIG. 2. Therefore, the observation position is obtained for each medical image 100 by specifying the center position of the tricuspid valve or the aortic valve by the user, or automatically detecting the center position as a characteristic point.

In a case where an observation position is detected automatically, the center position of the tricuspid valve or the aortic valve is detected as a characteristic point in the foregoing description, but the position of the center of gravity may be detected if the valve is not circular.

Further, the position P of the point where the apex of each valve intersects may be detected by detecting the shape of each valve constituting the tricuspid valve or the aortic valve. But, in this case, the position P cannot be clearly understood in a medical image 100 in which the three valves are fully open or in a medical image 100 in which the three valves are in the middle of opening, although the position P is clearly understood in a medical image 100 in which the three valves are completely closed.

Therefore, the position P may be obtained for a medical image 100 in which the three valves are fully open or for a medical image 100 in which the three valves are in the middle of opening through interpolation using the positions P detected from medical images 100 in phases before and after the phase of that medical image 100. Otherwise, the center positions or the positions of the centers of gravity of the tricuspid valves or the aortic valves may be detected automatically as described above for these medical images 100.

In the foregoing, a method of obtaining an observation position in a medical image 100 of a tricuspid valve or an aortic valve viewed from the above has been described, but not limited to this and an observation position may be obtained using a medical image 100 of these valves viewed from the side. For example, the position of an apex P (characteristic point) where each valve of an aortic valve intersects may be obtained as the observation position, as illustrated in FIG. 3. The same applies to a tricuspid valve. Any known method, such as pattern matching and the like may be used for detecting the apex P where each valve intersects.

The observation position of a tricuspid valve or an aortic valve is not limited to the position of the point where the apex of each valve intersects, and any other characteristic point having a characteristic shape may be designated or automatically detected as the observation position.

The clipping unit 40 clips each medical image 100 and extracts a clipped image of a certain range that includes the observation position described above from each medical image 100. The range and the shape of the clipping may be preset or may be designated by the user using the input device 2. In a case where the medical image 100 is a three-dimensional image, the clipping shape may include, for example, a rectangular solid, a sphere, a cylinder, and the like.

Further, when the clipping unit 40 performs clipping, a specific plane may be disposed in an image space and only the far side of the plane may be clipped. For example, in a case where a range that includes a tricuspid valve or an aortic valve is clipped, a clipped image of the tricuspid valve or the aortic valve observed from directly above is preferably generated. Therefore, it is only necessary to dispose a cutting plane above and near these valves perpendicularly to the blood passage of the tricuspid valve or the aortic valve and to clip only the far side of the cutting plane.

Here, an example of a method for clipping a range that includes a tricuspid valve in the clipping unit 40 will be described.

First, a set of medical images 100 in each phase is taken as V1, V2, V3, . . . , and Vn, and coordinates of the observation positions, i.e., the center positions of tricuspid valves in these medical images 100 are taken as X1, X2, X3, . . . , and Xn. In this case, the positional displacement between the position of the tricuspid valve of the medical image V1 in an initial phase and each of the positions of the tricuspid valves of the medical images in the other phases may be expressed by formulae given below:

D1=X2−X1

D2=X3−X1

D3=X4−X1

Dn−1=Xn−X1

Then, a clipping range of a cube centered on the central coordinate Xl is set in the medical image V1 in the initial phase. Next, when setting a clipping range in the medical image V2 in the next phase, setting a clipping range of a cube centered on the central coordinate X1, as in the medical image V1, will result in that the center position of the tricuspid valve of the medical image V2 is not located in the center position of the clipped image.

Therefore, when clipping the medical image V2, the center of the tricuspid valve can be located in the center of the clipped image by shifting the center of the clipping to X1+D1 (=X2). By shifting the center position of the clipping for the medical image V3 and subsequent medical images, as in the medical image V2, the center of the tricuspid valve can be placed in the center of each clipping image of medical images in all phases. Also, the aortic valve may be clipped in the same way as in the tricuspid valve.

The display control unit 50 performs an animation-display by successively displaying a set of clipped images clipped from each medical image 100 on the display 3.

Then, the display control unit 50 controls the animation-display of the clipped image clipped from each medical image 100 such that the observation position included in each clipped image is displayed at the same position on the display screen of the display 3. More specifically, in the case, for example, where the range that includes the tricuspid valve is clipped to generate a clipped image, as described above, the display control unit 50 performs control such that the center position of the tricuspid valve included in each clipped image is displayed at the same position on the display screen of the display 3.

The display position of the observation position in each clipped image may be preset or set and inputted by the user using the input device 2. In a case where the display position of the observation position is preset, the display position is preferably set to the center position of the display screen of the display 3.

In a case where the medical image 100 consists of volume data, the display control unit 50 displays a volume rendered or surface rendered clipped image on the display 3.

Further, in a case where the medical image 100 is a tomographic image, a two-dimensional tomographic image may be displayed on the display 3 as the clipped image. In the case where the two-dimensional tomographic image is displayed on the display 3, the two-dimensional tomographic image may have a thickness and, for example, a maximum intensity projection (MIP) image, a minimum intensity projection (MinIP) image, or a Raysum image may be displayed. The thickness of the two-dimensional image may be designated by the user using the input device 2.

Still further, in a case where the medical image 100 is a two-dimensional fluoroscopic radiation image, a clipped image of a predetermined rectangular range that includes the observation position in the two-dimensional fluoroscopic radiation image may be animation-displayed. The rectangular range may be preset or designated by the user using the input device 2.

The input device 2 is constituted by a pointing device, such as a keyboard, a mouse, or the like, to receive input of identification information of a display target medical image group 110 and input of clipping range and shape, as described above.

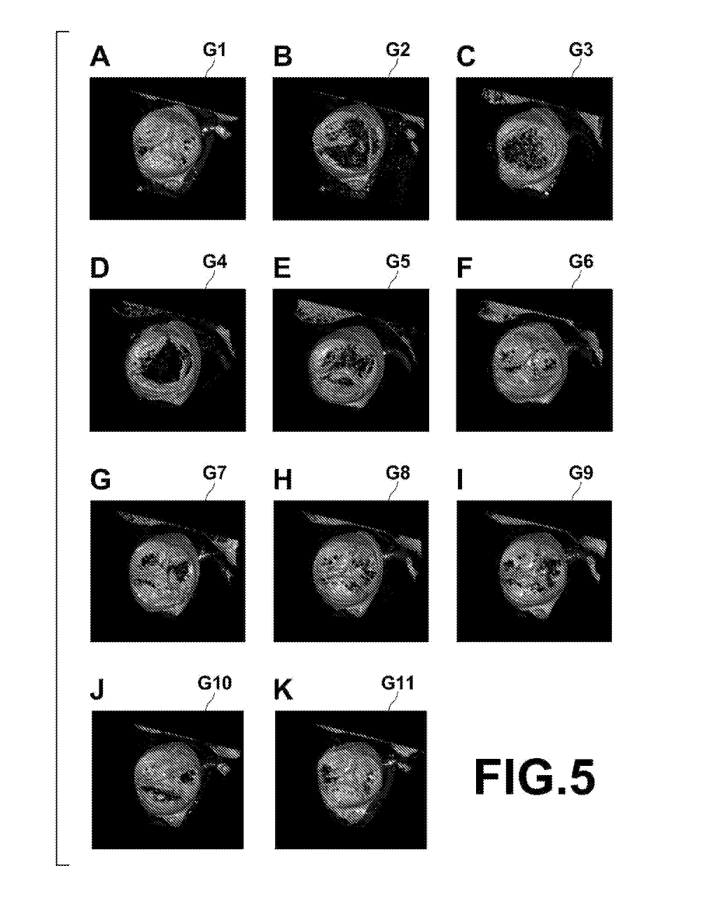

Next, an operation of the medical image diagnosis support system that uses the first embodiment of the present invention will be described with reference to the flowchart shown in FIG. 4.

First, subject identification information is inputted at the input device 2 and a medical image group 110 corresponding to the subject identification information is read out from the medical image storage unit 20 (S10).

Then, an observation position is obtained by the observation position obtaining unit 30 for each medical image 100 constituting the medical image group 110 read out from the medical mage storage unit 20 (S12).

Next, clipping range and shape are set and inputted by the user using the input device 2, and that information is inputted to the clipping unit 40 (S14). The clipping unit 40 clips each medical image 100 based on the inputted clipping range and shape, thereby generating a clipped image which includes the observation position.

Next, a display position of the observation position included in each clipped image on the display screen of the display 3 is set and inputted by the user using the input device 2 (S16).

Then, each clipped image and the display position of the observation position are inputted to the display control unit 50, and the display control unit 50 performs an animation-display by successively displaying each clipped image such that the observation position included in each clipped image is displayed at the same position on the display screen of the display 3 (S18).

FIG. 5 shows each clipped image, G1 to G11, extracted from a medical image 100 of an aortic valve viewed from the above. A to K of FIG. 5 show each clipped image, G1 to G11, arranged in time series, it can be seen that the aortic valve opens once from a closed state and returns to the closed state again. The successive display of each clipped image, G1 to G11, shown in A to K of FIG. 5 on the display 3 will result in an animation-display, thereby allowing the motion of the aortic valve to be observed.

According to the medical image diagnosis support system of the embodiment described above, the observation position included in each clipped image, i.e., the center position of the aortic valve is controlled so as to be displayed at the same position on the display screen of the display 3. This may cancel the movement of the aortic valve itself due to the beating of the heart and allows for an animation-display of only the motion of the aortic valve.

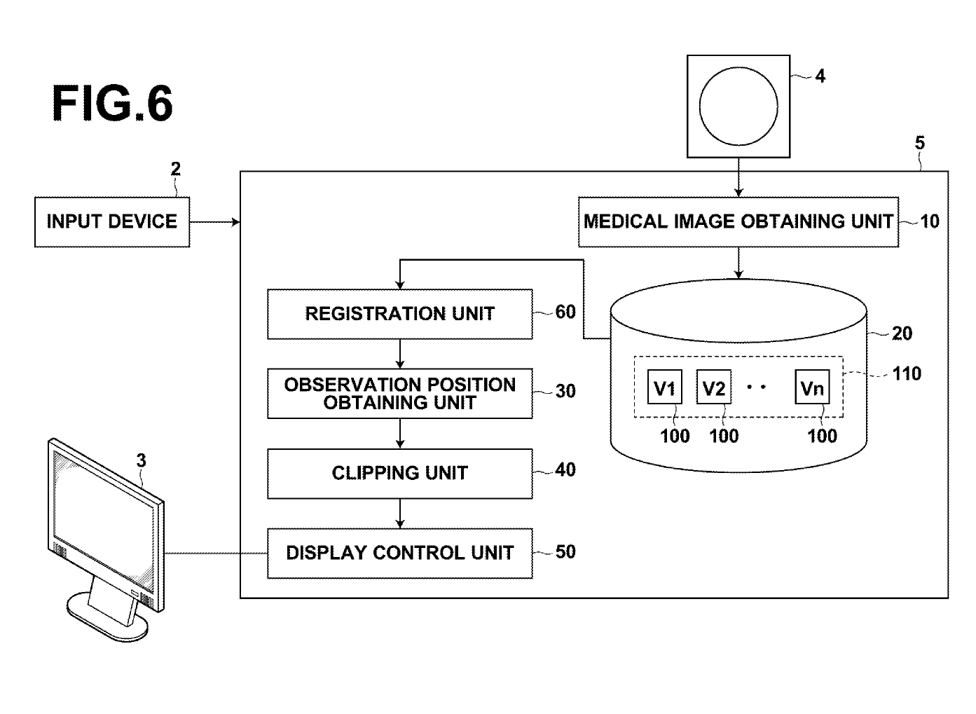

Next, a medical image diagnosis support system that uses a second embodiment of the present invention will be described. FIG. 6 is a block diagram of the medical image diagnosis support system that uses the present embodiment, illustrating a schematic configuration thereof.

While in the medical image diagnosis support system of the first embodiment described above, only one point in each medical image 100 is designated or automatically detected as the observation position, the medical image diagnosis support system of the second embodiment allows any point in a medical image 100 to be taken as the observation point.

More specifically, a medical image display control apparatus 5 of the medical image diagnosis support system of the second embodiment further includes a registration unit 60, as illustrated in FIG. 6.

The registration unit 60 performs a non-rigid entire image registration on a plurality of medical images 100 included in a medical image group 110 read out from the medical image storage unit 20 and obtains a correspondence relationship at any arbitrary point in each medical image 100. Any known method may be used for the non-rigid registration and the algorithms of the non-rigid registration may include, for example, those described in D. Rueckert et al., “Nonrigid Registration Using Free-Form Deformations: Application to Breast MR Images”, IEEE TRANSACTIONS ON MEDICAL IMAGING, Vol. 18, No. 8, pp. 712-721, 1999, Y. Wang and L. H. Staib, “Physical model-based non-rigid registration incorporating statistical shape information”, Medical Image Analysis, Vol. 4, No. 1, pp. 7-21, 2000, and PCT Japanese Publication No. 2005-528974.

The observation position obtaining unit 30 in the second embodiment obtains any position in an entire medical image 100 as an observation position.

More specifically, the observation position obtaining unit 30 receives designation of an observation position in one medical image 100 of the medical image group 110 and, based on the received observation position in the one medical image 100 and a correspondence relationship obtained as a result of the non-rigid registration, obtains an observation position in a medical image 100 other than the one medical image 100.

The observation position in the one medical image 100 is designated by the user using the input device 2.

Then, the clipping unit 40 of the second embodiment performs clipping with the observation position of each medical image 100 obtained in the observation position obtaining unit 30 in the center. The clipping range and shape are as in the first embodiment.

The display control unit 50 of the second embodiment performs an animation-display by successively displaying a set of clipped images clipped from each medical image 100 on the display 3, as in the first embodiment.

Also the display control unit 50 of the second embodiment controls the animation-display of the clipped image clipped from each medical image 100 such that the observation position included in each clipped image is displayed at the same position on the display screen of the display 3.

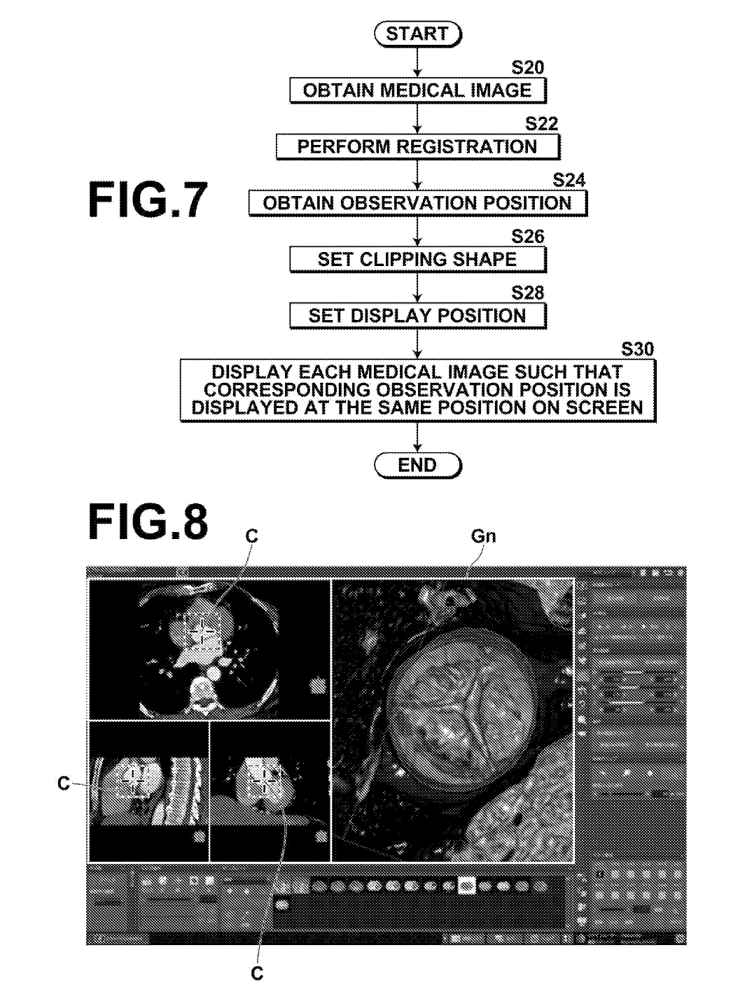

Next, an operation of the medical image diagnosis support system that uses the second embodiment of the present invention will be described with reference to the flowchart shown in FIG. 7.

First, as in the first embodiment, subject identification information is inputted at the input device 2 and a medical image group 110 corresponding to the subject identification information is read out from the medical image storage unit 20 (S20).

Then, the medical image group 110 read out from the medical image storage unit 20 is inputted to the registration unit 60, and the registration unit 60 performs a non-rigid entire image registration on each medical image included in the inputted medical image group 110 (S22).

Next, an observation position is obtained by the observation position obtaining unit 30 for each medical image 100 (S24). More specifically, in a case, for example, where each medical image 100 consists of volume data, a tomographic image generated from volume data of one medical image 100 is displayed on the display 3 as an observation position specifying image. The three tomographic images shown on the left of FIG. 8 are display examples of observation position specifying images described above.

FIG. 8 shows three tomographic images as observation position specifying images, but not necessarily three images and one tomographic image may be displayed, otherwise two or not less than four tomographic images may be displayed.

As shown in FIG. 8, a cross-hair cursor C for specifying an observation position is displayed on an observation position specifying image, and any position may be designated as an observation position by moving the cross-hair cursor C by the user using the input device 2. In a case where a plurality of tomographic images is displayed as observation position specifying images, as in FIG. 8, the cross-hair cursors C on the respective tomographic images are preferably moved interlocked with each other to indicate anatomically the same position.

After an observation position is designated for one medical image 100, a corresponding observation position in a medical image 100 other than the one medical image 100 is obtained based on the observation position of the one medical image 100 and a result of the non-rigid registration.

Next, clipping range and shape are set and inputted by the user using the input device 2, and that information is inputted to the clipping unit 40 (S26). The clipping unit 40 performs clipping with the observation position of each medical image 100 in the center based on the inputted clipping range and shape, thereby generating a clipped image.

Next, a display position of the observation position included in each clipped image on the display screen of the display 3 is set and inputted by the user using the input device 2 (S28).

Then, each clipped image and the display position of the observation position are inputted to the display control unit 50, and the display control unit 50 performs an animation-display by successively displaying each clipped image such that the observation position included in each clipped image is displayed at the same position on the display screen of the display 3 (S30). The image displayed on the right of the three tomographic images in FIG. 8 is a clipped image On to be animation-displayed. It is preferable that an observation position specifying image and a clipped image are displayed at the same time, as in FIG. 8.

According to the medical image diagnosis support system of the second embodiment, any point in a medical image 100 may be designated as an observation position. This may cancel the movement of the designated observation position itself and allows for an animation-display of only the motion near the observation position.

Note that, in the second embodiment, the observation position may also be changed in real time.

In the medical image diagnosis support systems of the first and the second embodiments, each clipped image may be displayed such that a relative position between a range clipped by the clipping unit 40 and the observation position described above is maintained.

Further, in the medical image diagnosis support systems of the first and the second embodiments, a certain image range which includes an observation position is extracted from each medical image by the use of clipping, but a specific area which includes an observation position may be extracted manually or automatically without using the clipping. More specifically, in a case, for example, where the observation position is an aortic valve, an aortic arch region that includes the aortic valve may be extracted manually or automatically and only the aortic arch region may be animation-displayed. In this case also, the observation position included in the aortic arch region of each medical image 100 is controlled so as to be displayed at the same position on the display screen of the display 3.

Still further, in the medical image diagnosis support systems of the first and the second embodiments, a set of medical images may be successively displayed such that the foregoing observation position does not move in a depth direction of the display screen.

What is claimed is:

1. A medical image display control apparatus, comprising:
a medical image obtaining unit that obtains a set of time series medical images captured by successively imaging the same subject;
an observation position obtaining unit that obtains anatomically common positions in the set of medical images as observation positions; and
a display control unit that successively displays the set of medical images such that the observation positions in the set of medical images are displayed at the same position on a display screen.

2. The medical image display control apparatus of claim 1, wherein the observation position obtaining unit receives designation of the observation positions in the set of medical images.

3. The medical image display control apparatus of claim 1, wherein the observation position obtaining unit automatically detects the observation positions in the set of medical images.

4. The medical image display control apparatus of claim 1, wherein the apparatus comprises a registration unit that performs a non-rigid registration on the set of medical images.

5. The medical image display control apparatus of claim 4, wherein the observation position obtaining unit receives designation of an observation position in one of the set of medical images and, based on the received observation position in the one medical image and a result of the non-rigid registration, obtains an observation position in a medical image other than the one medical image.

6. The medical image display control apparatus of claim 1, wherein the display control unit successively displays the set of medical images such that the observation positions in the set of medical images are displayed in the center position of the display screen.

7. The medical image display control apparatus of claim 1, wherein:
the medical image is an image obtained by imaging a heart, including a valve;
the observation position obtaining unit obtains an observation position in the valve; and
the display control unit displays the medical image of the valve viewed from the above.

8. The medical image display control apparatus of claim 1, wherein the apparatus comprises a clipping unit that clips the set of medical images.

9. The medical image display control apparatus of claim 8, wherein the clipping unit receives a clipping shape and performs the clipping in the received shape.

10. The medical image display control apparatus of claim 8, wherein the clipping unit disposes a specific plane in an image space and clips only the far side of the plane.

11. The medical image display control apparatus of claim 1, wherein the display control unit displays a volume rendered or a surface rendered image as the medical image.

12. The medical image display control apparatus of claim 1, wherein the display control unit displays a two-dimensional tomographic image as the medical image.

13. The medical image display control apparatus of claim 12, wherein the two-dimensional tomographic image has a thickness.

14. The medical image display control apparatus of claim 13, wherein the two-dimensional tomographic image is a MIP image, a MinIP image, or a Raysum image.

15. The medical image display control apparatus of claim 1, wherein the medical image is an image captured by a CT system or a MR system.

16. The medical image display control apparatus of claim 8, wherein the display control unit successively displays the set of medical images such that a relative position between a range clipped by the clipping unit and the observation position is maintained.

17. The medical image display control apparatus of claim 8, wherein the display control unit successively displays the set of medical images such that the observation positions do not move in a depth direction of the display screen.

18. A medical image display control method, comprising the steps of:
obtaining a set of time series medical images captured by successively imaging the same subject;
obtaining anatomically common positions in the set of medical images as observation positions; and
successively displaying the set of medical images such that the observation positions in the set of medical images are displayed at the same position on a display screen.

19. A non-transitory computer-readable recording medium containing a medical image display control program for causing a computer to function as:
a medical image obtaining unit that obtains a set of time series medical images captured by successively imaging the same subject;
an observation position obtaining unit that obtains anatomically common positions in the set of medical images as observation positions; and
a display control unit that successively displays the set of medical images such that the observation positions in the set of medical images are displayed at the same position on a display screen.
